(19) United States
(12) Patent Application Publication
(10) Pub. No.: US 2009/0043398 A1
(43) Pub. Date: Feb. 12, 2009

(54) METHOD OF PRODUCING GRADIENT ARTICLES BY CENTRIFUGATION MOLDING OR CASTING

(75) Inventors: Donald L. Yakimicki, Warsaw, IN (US); Brian H. Thomas, Columbia City, IN (US)

Correspondence Address: WOOD, HERRON & EVANS, LLP (ZIMMER), 2700 CAREW TOWER, 441 VINE STREET, CINCINNATI, OH, 45202, US

(73) Assignee: ZIMMER, INC., Warsaw, IN (US)

(21) Appl. No.: 11/836,480

(22) Filed: Aug. 9, 2007

(51) Int. Cl. A61F 2/28

(52) U.S. Cl. 623/23.51; 264/271.1; 264/311

(57) ABSTRACT

The present invention provides a method for producing articles with a gradient of density, porosity and/or concentration by subjecting a viscous material to centrifugation during production of the article. The viscous material may be a composite material comprising a hydrogel. The viscous material can be molded or cast into the article. In certain embodiments, the viscous material is used to create an articulating surface implant such as a replacement plug, a knee spacer, or a spinal disc. The article may also be an implant such as a shoulder implant or other socket type implant that is produced by centrifuging in two axes which produces a gradient relative to both axes of rotation.

### FIELD OF THE INVENTION

The present invention relates generally to methods for producing articles comprising a gradient and specifically, to a gradient in density, porosity, or concentration provided by centrifugation.

### BACKGROUND

Hydrogels are water-swellable or water-swollen materials whose structure is typically defined by a crosslinked or interpenetrating network of hydrophilic homopolymers or copolymers. The hydrophilic homopolymers or copolymers can be water-soluble in free form, but in a hydrogel they may be rendered insoluble generally due to the presence of covalent, ionic, or physical crosslinks. In the case of physical crosslinking, the linkages can take the form of entanglements, crystallites, or hydrogen-bonded structures. The crosslinks in a hydrogel provide structure and physical integrity to the polymeric network.

Hydrogels can be classified as amorphous, semicrystalline, hydrogen-bonded structures, supermolecular structures, or hydrocolloidal aggregates. Numerous parameters affect the physical properties of a hydrogel, including porosity, pore size, nature of gel polymer, molecular weight of gel polymer, and crosslinking density. The crosslinking density influences the hydrogel's macroscopic properties, such as volumetric equilibrium swelling ratio, compressive modulus, or mesh size. Pore size and shape, pore density, and other factors can impact the surface properties, optical properties, and/or mechanical properties of a hydrogel.

Hydrogels have been fabricated from a variety of hydrophilic polymers and copolymers. Poly(vinyl alcohol), poly(ethylene glycol), poly(vinyl pyrrolidone), polyacrylamide, and poly(hydroxyethyl methacrylate), and copolymers of the foregoing, are examples of polymers from which hydrogels have been made.

Over the past three to four decades, hydrogels have shown promise for biomedical and pharmaceutical applications, mainly due to their high water content and rubbery or pliable nature, which can mimic natural tissue. An additional advantage of hydrogels is that they may provide desirable protection of drugs, peptides, and proteins from the potentially harsh environment in the vicinity of a release site. Thus, such hydrogels could be used as carriers for the delivery of proteins or peptides by a variety of means, including oral, rectal, or in situ placement. Transport of eluents either through or from a hydrogel is affected by pore size and shape, pore density, nature of polymer, degree of hydration, and other factors. Also, hydrogels have been widely employed in the fabrication of contact lenses and can be made to have properties similar to cartilage, therefore, hydrogels are one of the most promising materials for meniscus and articular cartilage replacement.

### SUMMARY OF THE INVENTION

The present invention provides a method for producing an article with a gradient of density, porosity and/or concentration by subjecting a viscous material to centrifugation during production. The viscous material may be a composite material comprising at least a first and a second constituent. The centrifugal force of the present invention causes movement of the first constituent of the viscous material relative to the second constituent. The viscous material can be molded or cast into an article either before, during, or after subjecting the viscous material to centrifugation. The movement of the first constituent relative to the second constituent creates a gradient in the resulting article.

In another embodiment, the present invention provides for a polymeric composite implant comprising a gradient of at least one of density, porosity, or concentration produced by a centrifugal force. The gradient is formed between a point distal to an axis of rotation and a point proximal to the axis of rotation. In some embodiments, the gradient in the implant is formed relative to more than one axes of rotation.

In another embodiment, the present invention provides for a hydrogel implant comprising a gradient in stiffness produced by a centrifugal force. The gradient is formed between a point distal to an axis of rotation and a point proximal to the axis of rotation. In some embodiments, the gradient in the implant is formed relative to more than one axes of rotation.

### BRIEF DESCRIPTION OF THE FIGURES

FIG. 1 shows a glenoid structure formed according to one embodiment of the invention where a viscous material is subjected to centrifugation in two axes of rotation.

FIG. 1A shows a cross-sectional view of FIG. 1 along line 1A.

FIG. 2 shows an example of rotation about two axes in one embodiment of the invention.

FIG. 3 shows an articulating surface replacement plug according to one embodiment of the invention.

FIG. 4 shows a replacement spinal disc according to one embodiment of the invention.

FIG. 5 shows a replacement knee component according to one embodiment of the invention.

### DETAILED DESCRIPTION

The present invention provides a method of producing an article from a viscous material whereby the article exhibits a gradient of at least one of density, porosity, or concentration. The method comprises subjecting the material in viscous form to a centrifugal force, and casting or molding the material, to thereby form an article exhibiting the gradient. The inventive method utilizes the application of centrifugal force to achieve separation of the constituents of the material based on a property of the material affected by centrifugal force, such as density, concentration, or porosity. This present invention provides for articles having different properties at different surfaces or at different depths within the article. In one embodiment, casting is accomplished by spin casting. Spin casting is a method of utilizing centrifugal force to produce castings from a mold. Typically, the casting material is poured in through an opening at the top-center of the mold and the filled mold then continues to spin as the casting material sets. In one embodiment, the viscous material comprises a polymer dispersed in a solvent. The viscous material may be at elevated temperatures such as the case with a thermoplastic lyogel. A viscous material exhibiting a density gradient may then be formed using the inventive method based on characteristics of the polymer, such as differences in molecular weight or branched/unbranched polymer chains.

In another embodiment, the viscous material comprises a hydrogel precursor or water-swellable material precursor. As a result of the inventive method, the resulting article is a hydrogel or water-swellable material exhibiting a gradient in, for example, density, concentration, or porosity relative to the axis of rotation. In another embodiment, the viscous material may be a composite material comprising at least a first and a second constituent.

In some embodiments, the viscous material is subjected to rotation, and thus centrifugal force, about more than one axis. Subjecting the viscous material to rotation about more than one axis of rotation results in the formation of a gradient relative to each axis of rotation. The gradient may be formed based on a property of the viscous composition such as density, concentration, or porosity.

In one embodiment, a viscous material is subjected to the inventive method to create a density gradient relative to an axis of rotation. In one embodiment, the viscous material comprises a first constituent of a greater density than a second constituent. In another embodiment, the first constituent is a polymeric material and the second constituent is a solvent. Following application of an effective amount of centrifugal force, the first constituent is moved away from an axis of rotation and results in an article exhibiting an increasing density away from the axis of rotation.

In one embodiment, the viscous material comprises a first polymeric matrix constituent and a second particulate or fibrous dispersed constituent. Subjecting the viscous material to the centrifugal force is effective to cause movement of the second particulate or fibrous dispersed constituent away from an axis of rotation whereby the article exhibits an increasing concentration gradient of the second particulate or fibrous dispersed constituent in a direction away from the axis of rotation. Examples of particles or nanoparticles that may be included in the viscous material include barium sulfate and zirconium dioxide. In some embodiments, the presence of particles in the article provides an increasing stiffness gradient to the material and/or imparts radiopacity. Other examples of particulate material that may be included in the viscous material are clays, fibrin, collagen, ceramics, and nanotubes. Examples of fibers that may be included in the viscous material are carbon fibers, fibers formed from ultra high molecular weight polyethylene, such as Spectra® (Honeywell), polyurethane, acrylic, nylon, PEEK, polyacrylamide, polyethylene-co-vinyl alcohol, and poly vinyl alcohol (PVA). Other examples of fibrous material that may be included in the viscous material include glass or ceramic fibers, for example calcium phosphate fibers. In one embodiment, the viscous material is formed of PVA and PVA fibers.

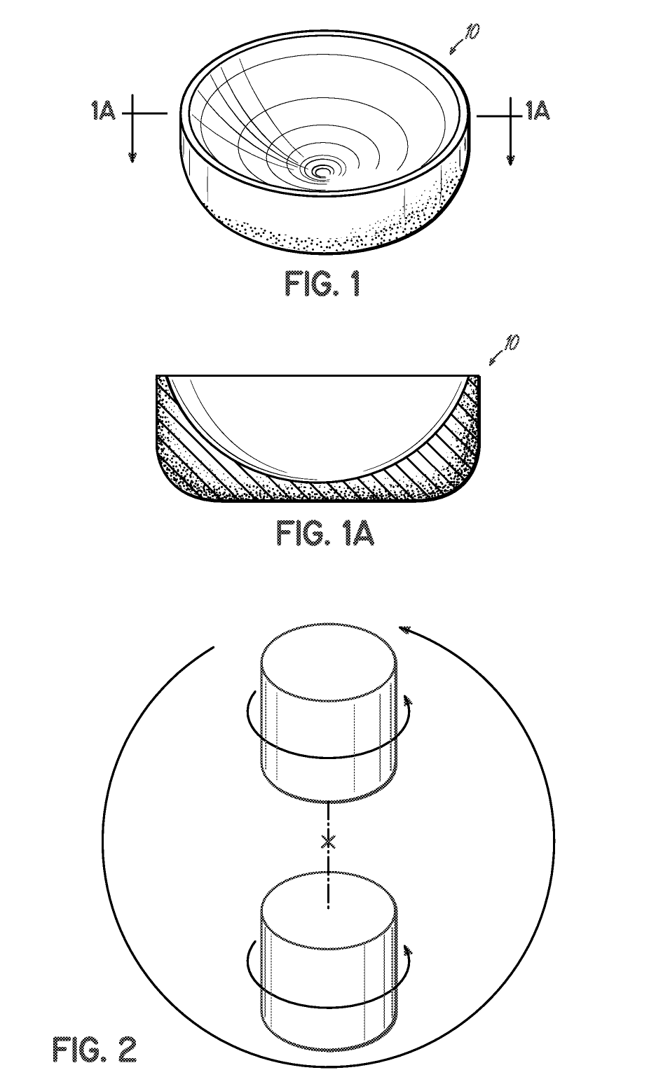

In one embodiment, the viscous material is rotated in more than one axes of rotation. The rotation about more than one axes of rotation may occur concurrently or sequentially. The multiple axes of rotation results in an article with an increasing density gradient in the direction away from each of the more than one axes of rotation. In one embodiment, the rotation relative to more than one axes of rotation results in a glenoid-shaped structure (10), as shown in FIG. 1. Cross-section of the glenoid-shaped structure is shown in FIG. 1A. In one embodiment, the viscous material is subjected to two axes of rotation as shown in FIG. 2 where the viscous material is rotated about its center axis and rotated relative to an external point.

In one embodiment, the viscous material is porous such that one constituent of the material is a plurality of pores. Subjecting the viscous material to the centrifugal force is effective to cause movement of the pores toward an axis of rotation whereby the article exhibits an increasing porosity gradient in a direction toward the axis of rotation. In one embodiment, the viscous material is rotated in more than one axes of rotation to provide the increasing porosity gradient in the direction toward each of the more than one axes of rotation.

Centrifugation may be accomplished by any of a variety of centrifuges that are available and are known to one skilled in the art. By way of example only and not limitation, a Beckman Optima™ LE Ultracentrifuge, which has a maximum speed of 80,000 rpm and a maximum force of 602,000× gravity (g), may be used, or a Zimmer Bone Cement Centrifuge (model 5069-02) may be used. Commercially available spin casting equipment such as the Contenti ECM120, which has a maximum speed of 1,000 rpm and a maximum force of 341× gravity (g), or the Nicem® C500 which has a maximum speed of 1,500 rpm and maximum force of 1,152× gravity (g) may be used for the filling of molds, in addition to centrifugation.

The viscous material may be shaped into a variety of three dimensional forms such as cylindrical derivatives or segments, spherical derivatives or segments, or polyhedral derivatives or segments. Suitable shapes may include at least one cylindrical, spherical or polyhedral segment. Complex shapes that may include combinations of cylindrical, spherical and/or polyhedral shapes are also within the scope of the present invention. In one embodiment, the viscous material is shaped in a tapered oval.

Processing methods to obtain a resulting article of desired shape or size may include solution casting, injection molding, or compression molding. In general, these methods may be used before or after crosslinking, as well as before or after the article is hydrated, in the case of water-swellable materials.

To prepare a viscous material for use in casting, the appropriate polymers (and optionally any additives) are dissolved in the solvent. Heating the solvent may assist in dissolution of the polymers. The polymer-to-solvent ratio can vary widely. PVA hydrogels, by way of illustration, have reportedly been prepared using a polymer concentration of 2 to 70% by weight using a variety of solvents including water, dimethyl sulfoxide, or a combination thereof.

To prepare a viscous material for compression or injection molding, the appropriate polymers (and optionally any additives) can be compounded in a heated mixing device such as a twin-screw compounder with the appropriate diluent or plasticizer. Heating the mixing device may assist in processing. Suitable temperatures depend on diluent or plasticizer and the chosen polymer system. The polymer-to-diluent ratio can vary widely.

In one embodiment, the viscous material may be first subjected to centrifugal force to form the gradient, and then cast or molded into an article. In other embodiments, the casting or molding of the viscous material may occur prior to or during centrifugation according to the inventive method.

Optionally, the viscous material, the polymeric composite material, the hydrogel, or articles of the present invention may be subjected to one or more crosslinking steps. Crosslinking may be carried out after forming the gradient in the viscous material, after shaping the material into an article, or at any other suitable point during processing. A variety of conventional approaches may be used to crosslink the composite material, including, physical crosslinking (e.g., freeze thaw method), photoinitiation, irradiation and chemical crosslinking.

The inventive article formed from a viscous material and subjected to centrifugation can be used in a variety of applications, including minimally invasive surgical procedures, as known in the field. By way of example, the viscous material can be used to provide artificial articular cartilage implants. In one embodiment, the viscous material of the present invention is used to form an artificial meniscus or articular bearing components. In another embodiment, the viscous material of the present invention is used to form implants employed in temporomandibular joints, in proximal interphalangeal joints, in metacarpophalangeal joints, in metatarsalphalanx joints, or in hip capsule joint repairs.

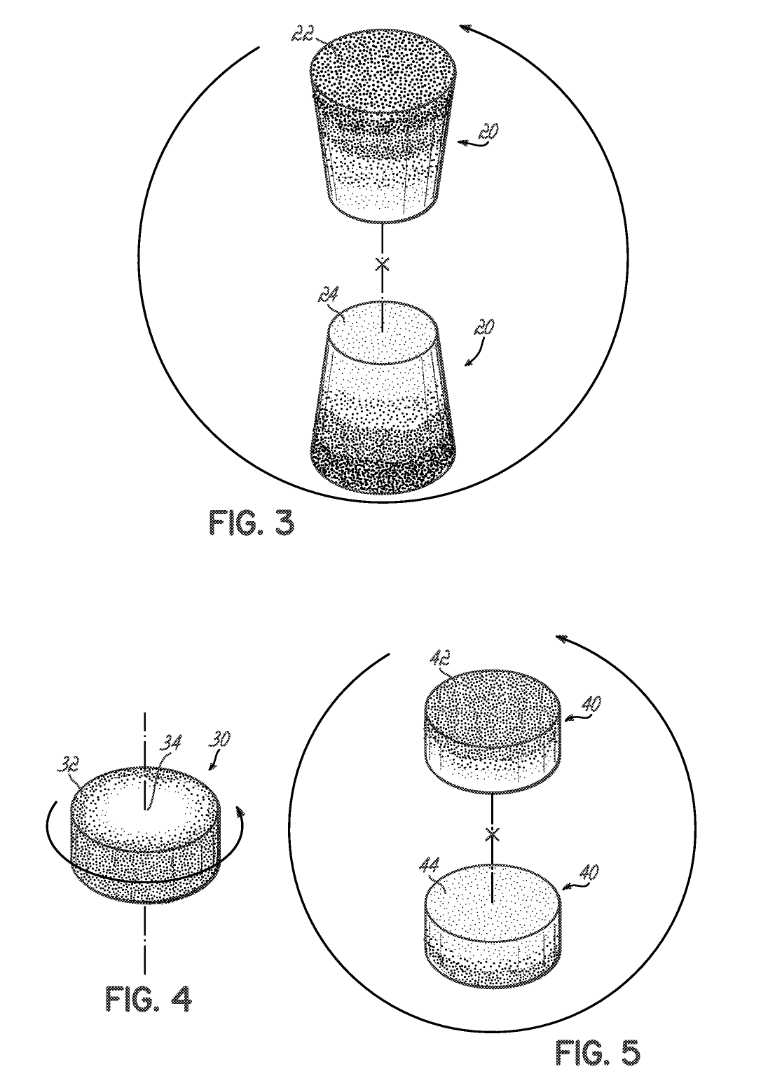

In the case of an articulating surface implant, the article would have a gradient of stiffness transitioning from a stiffer material at the bone interface for fixation to a less stiff material at the articulating surface. In certain embodiments, the bone interface surface may incorporate a porous metal base. In one embodiment, the article is an articulating surface replacement plug (20) as shown in FIG. 3, having an oval tapered geometry, a bone-contacting end (22), an articulating end (24), and a gradient formed by the inventive method. The oval tapered geometry is designed to be pressed into a mating cavity and prevents rotation or displacement. In one embodiment, the gradient formed within the article provides graded stiffness ranging from increased stiffness at the bone-contacting end (22) to decreased stiffness at the articulating end (24). The stiffness is created by a property of the viscous material such as density, concentration, or porosity. In one embodiment, a porous metal or woven base is attached to the bone-contacting end (22) of the plug.

In another embodiment, the article formed from the inventive method is a replacement spinal disc (30), as shown in FIG. 4. Degenerative disc disease in the lumbar spine is marked by a dehydration of the intervertebral disc and loss of biomechanical function of the spinal unit. The viscous material of the present invention can also be employed in a spinal disc implant used to replace a part or all of a natural human spinal disc. The resulting spinal disc implant has a graded stiffness ranging from increased stiffness at a periphery of the disc (32) to decreased stiffness in a center of the disc (34).

In another embodiment, the article formed from the inventive method is a replacement knee component (40) having a bone-contacting end (42) and an articulating end (44), as shown in FIG. 5. The resulting knee component has a graded stiffness ranging from increased stiffness at the bone-contacting end (42) to decreased stiffness at the articulating end (44).

The present invention also provides for a polymeric composite implant comprising a gradient of at least one of density, porosity, or concentration. The gradient in at least one of density, porosity, or concentration results from a centrifugal force applied to the composite material. The resulting gradient is formed between a point distal to an axis of rotation and a point proximal to the axis of rotation.

The present invention also provides for a hydrogel implant comprising a gradient in stiffness. The gradient is produced by subjecting a hydrogel precursor to a centrifugal force. The resulting gradient is formed between a point distal to an axis of rotation and a point proximal to the axis of rotation.

Numerous materials, as described below, may be used to form the first and second constituents making up the viscous material. Particularly, the viscous material may comprise a polymer. Examples of polymers that may be used in the invention include polyurethane, polyethylene, polyetheretherketone (PEEK), and acrylic. In one embodiment, the first and second constituents are the same type of polymer but differ in an intrinsic physical parameter such as molecular weight. For instance, the first and second constituents may be the same polymer but have different chain lengths or a different amount of chain branching. In another embodiment, one of the constituents may not be a polymeric material and may be, for instance, a solvent. In some embodiments, the viscous material comprises a hydrogel or water-swellable material. Further examples of suitable materials to be used in the viscous material can be found in U.S. patent application Ser. No. 11/614,389, incorporated by reference herein in its entirety.

Polymeric materials that may be used to make the viscous material include water-swellable materials and hydrogels and typically include a hydrophilic polymer. In one embodiment, the hydrophilic polymer may be poly vinyl alcohol (PVA), or derivatives thereof. By way of illustration only, other hydrophilic polymers that may be suitable include polyhydroxyethyl methacrylate, polyvinyl pyrrolidone, polyacrylamide, polyacrylic acid, hydrolyzed polyacrylonitrile, polyethyleneimine, ethoxylated polyethyleneimine, polyallylamine, or polyglycols as well as blends or mixtures of any of these hydrophilic polymers. In certain embodiments, at least one component of the hydrogel is PVA as the hydrophilic polymer.

In some embodiments of the present invention, the hydrophilic polymer may be a hydrogel blend including PVA and a second polymer having hydrophobic recurring units and hydrophilic recurring units. The second polymer may be polyethylene-co-vinyl alcohol, for example. As non-limiting examples, other suitable polymers include diol-terminated polyhexamethylene phthalate and polystyrene-co-allyl alcohol.

Hydrogels possess a unique set of mechanical properties. In certain embodiments, such as the blended hydrogel described above, these materials exhibit toughness comparable or superior to other hydrogels including PVA-based hydrogels, while maintaining flexibility and a low elastic modulus. Examples of these improved properties are increased tensile strength, increased shear resistance, and improved elasticity. Furthermore, the properties of the blended hydrogels can be tailored to meet the requirements for a specific usage. Additionally, following the inventive method, the properties of the hydrogels can be gradated, for example, by having increased stiffness away from an axis of rotation.

The article of the present invention may also include additional polymers, peptides and proteins, such as collagen, or conventional additives such as plasticizers, components for inhibiting or reducing crack formation or propagation, components for inhibiting or reducing creep, or particulates or other additives for imparting radiopacity to the article. By way of example only, an additive for imparting radiopacity can include metal oxides, metal phosphates, and metal sulfates such as barium sulfate, barium titanate, zirconium oxide, ytterbium fluoride, barium phosphate, and ytterbium oxide. Biopolymers may also be used in certain embodiments. Suitable biopolymers include anionic biopolymers such as hyaluronic acid, cationic biopolymers such as chitosan, amphipathic polymers such as collagen, gelatin and fibrin, and neutral biopolymers such as dextran and agarose. Optionally, additives such as biocompatible preservatives, surfactants, colorants and/or other additives conventionally added to polymer mixtures may be included in the inventive article.

In one embodiment where the viscous material contains a hydrogel, the hydrogel may be used to release therapeutic drugs or other active agents. Hydrogels can be suitably employed in vivo to provide elution of a protein, drug, or other pharmacological agent impregnated in the hydrogel or provided on the surface of the hydrogel.

An embodiment of a composite material that may be used in the present invention is set out in the following example.

Crosslinked PVA fibers were added to a solution of PVA in DMSO at a temperature of 80° C. and were mixed. Following cooling, the gel-like composite material was subjected to centrifugation at 2,500 rpm for 1 minute in a Zimmer Bone Cement Centrifuge (model 5069-02). The rotor containing the composite material had a radius of 7.5″, which translates to a centrifugal force of approximately 1,330×g.

The resulting composite material exhibited a gradient of increasing concentration of the PVA fibers moving away from the axis of rotation with a soft, smooth texture toward the axis of rotation transitioning to a harder, rougher texture away from the axis of rotation.

The invention is further set forth in the claims listed below. This invention may take on various modifications and alterations without departing from the scope thereof. In describing embodiments of the invention, specific terminology is used for the sake of clarity. The invention, however, is not intended to be limited to the specific terms so selected, and it is to be understood that each term so selected includes all technical equivalents that operate similarly.

What is claimed is:

1. A method of producing an article from a viscous material comprising a first constituent and a second constituent, the method comprising subjecting the viscous material to a centrifugal force to cause movement of the first constituent relative to the second constituent, and casting or molding the viscous material, whereby an article is formed exhibiting a gradient of at least one of density, porosity, or concentration, and wherein the casting or molding of the viscous material occurs either before, during, or after subjecting the viscous material to the centrifugal force.

2. The method of claim 1 wherein at least one of the first constiuent or the second constiuent is a hydrogel.

3. The method of claim 1 wherein the first constituent has a greater density than the second constituent, and wherein the centrifugal force is effective to cause movement of the first constituent away from an axis of rotation whereby the article exhibits an increasing density gradient in a direction away from the axis of rotation.

4. The method of claim 3 wherein the viscous material is rotated in more than one axes of rotation to provide the increasing density gradient in the direction away from each of the more than one axes of rotation.

5. The method of claim 1 wherein the second constituent is a polymeric material and the first constituent is a particulate or fibrous material, and wherein subjecting the viscous material to the centrifugal force is effective to cause movement of the particulate or fibrous material away from an axis of rotation whereby the article exhibits an increasing concentration gradient of the particulate or fibrous material in a direction away from the axis of rotation.

6. The method of claim 5 wherein the viscous material is rotated in more than one axes of rotation to provide the increasing concentration gradient in the direction away from each of the more than one axes of rotation.

7. The method of claim 1 wherein the viscous material is porous such that the first constituent is a plurality of pores, and wherein subjecting the viscous material to the centrifugal force is effective to cause movement of the plurality of pores toward an axis of rotation whereby the article exhibits an increasing porosity gradient in a direction toward the axis of rotation.

8. The method of claim 7 wherein the viscous material is rotated in more than one axes of rotation to provide the increasing porosity gradient in the direction toward each of the more than one axes of rotation.

9. The method of claim 1 wherein the article is an articulating surface replacement plug having an oval tapered geometry, a bone-contacting end, and an articulating end, and wherein the gradient provides graded stiffness ranging from increased stiffness at the bone-contacting end to decreased stiffness at the articulating end.

10. The method of claim 9 further comprising adding a porous metal or woven base to the bone-contacting end of the plug.

11. The method of claim 1 wherein the article is a replacement spinal disc, and wherein the gradient provides graded stiffness ranging from increased stiffness at a periphery of the disc to decreased stiffness in a center of the disc.

12. The method of claim 1 wherein the article is a replacement knee component having a bone-contacting end, and an articulating end, and wherein the gradient provides graded stiffness ranging from increased stiffness at the bone-contacting end to decreased stiffness at the articulating end.

13. A polymeric composite implant comprising a gradient of at least one of density, porosity, or concentration wherein the gradient results from a centrifugal force whereby the gradient is formed between a point distal to an axis of rotation and a point proximal to the axis of rotation.

14. A hydrogel implant comprising a gradient in stiffness, wherein the gradient is produced by subjecting a hydrogel precursor to a centrifugal force whereby the gradient is formed between a point distal to an axis of rotation and a point proximal to the axis of rotation.
